(19) United States
(12) Patent Application Publication
(10) Pub. No.: US 2026/0338799 A1
(43) Pub. Date: Oct. 29, 2026

(54) IN VITRO METHOD FOR PREDICTING ORGAN TRANSPLANT REJECTION

(71) Applicants: FUNDACIÓN PARA LA FORMACIÓN E INVESTIGACIÓN SANITARIA DE LA REGIÓN DE MURCIA, El Palmar (Murcia) (ES); BRAVO INNOVATION, Madrid (ES)

(72) Inventors: Sergio CAJA GALÁN, Madrid (ES); Alberto BAROJA MAZO, El Palmar (Murcia) (ES)

(21) Appl. No.: 19/115,222

(22) PCT Filed: Sep. 25, 2023
(86) PCT No.: PCT/EP2023/076351

§ 371 Date: Mar. 25, 2025

Related U.S. Application Data
(60) Provisional application No. 63/377,168, filed on Sep. 26, 2022.

(51) Int. Cl. G01N 33/68

(52) U.S. Cl. CPC G01N 33/6893; G01N 2800/245

(57) ABSTRACT

The present invention refers to an in vitro method for predicting organ transplant rejection, preferably acute organ transplant rejection, most preferably acute liver transplant rejection, in a subject in need thereof.

### FIELD OF THE INVENTION

The present invention refers to the medical field. Particularly, the present invention refers to an in vitro method for predicting organ transplant rejection, preferably acute organ transplant rejection, most preferably acute liver transplant rejection, in a subject in need thereof.

### STATE OF THE ART

Deceased donor liver transplantation is nowadays a routine procedure for the treatment of terminal liver failure and often represents the only chance of a cure. Although donation after brain death (DBD) account for the bulk of organ donors, in the recent years there has been a growing interest in increasing the donor pool. As a result, organ donation after circulatory death (DCD) has been reintroduced and contributes to increasing the number of donations in many countries. These donations currently account for 32% of donors in Spain, and it has been one of the keys applied to reach the number of 46 donors/million inhabitants in 2017.

Between 30 and 40% of transplanted patients will suffer some episode of acute rejection or early graft dysfunction in the first weeks after transplantation. However, the occurrence of acute rejection episodes increases by 40% the probability of suffering chronic rejection and loss of the transplant in the medium term. In addition, up to 4% of these patients lose the organ in the first weeks, leading to the need for a new emergency re-transplantation, which cannot always be carried out. All this puts the life of the patients at risk and also determines that 30% of the patients end up dying from rejection-related causes. In addition, the identification of a rejection episode is done when it is occurring. To date, there is no tool that allows early prediction of the appearance of these episodes and their serious consequences for patient health. So, there is an unmet medical need of finding reliable methods for predicting organ transplant rejection, which may help clinicians to select, in advance, possible treatments that could be needed, thus improving the chances of treatment success.

The present invention is thus focused on solving this problem, and a new method for predicting organ transplant rejection, preferably acute organ transplant rejection, most preferably acute liver transplant rejection, is herein provided.

### DESCRIPTION OF THE INVENTION

### Brief Description of the Invention

As cited above, the present invention refers to a method for predicting organ transplant rejection, preferably acute organ transplant rejection, most preferably acute liver transplant rejection, in a subject in need thereof, which comprises assessing the concentration or expression level of specific biomarkers, wherein the identification of a variation or deviation (increase or decrease) in the concentration or expression level with respect to a pre-established threshold level determined in control subjects, is an indication that the subject may suffer organ transplant rejection.

Particularly, the inventors assayed the biomarkers shown in Table 1 and Table 2. Then, the biomarkers were quantified such as it is shown in Table 5 and Table 6. Interestingly, such as it is shown in Table 8 and Table 9, several combinations of at least two of the assayed biomarkers are associated with the prediction of organ transplant rejection showing high ranked AUC values, always above 0.7.

Such as it is shown in FIG. 1, FIG. 2 and FIG. 3, there are clear differences between each marker when comparing the non-rejection group and the acute rejection group. In a preferred embodiment, the following combination of biomarkers were assayed: Fibronectin, Hyaluronic acid, MIP1B, mitDNA, MMP7, MMP8, Angiogenin and ANGPT2, giving rise to a Sensitivity and Specificity of 99.6% (see FIG. 4).

This means that any individual biomarker of Table 1 and Table 2, or any combination thereof, comprising from at least two biomarkers to all the biomarkers of Table 1 and Table 2, (i.e., three, four, five . . . until all the biomarkers of Table 1 and Table 2) may be used, according to the present invention to predict organ transplant rejection in a subject in need thereof.

So, the first embodiment of the present invention refers to an in vitro method (hereinafter method of the invention) for predicting organ transplant rejection in a subject in need thereof which comprises: a) Assessing the concentration or expression level of at least a biomarker selected from the group consisting of: Fibronectin (FIBRONECT), Hyaluronic (HYALURON), MIP1B, HLADR_FCM, mitDNA, MMP-7, MMP-8, Angiogenin, Angiopoietin-2, Thrombospondin-1, MMP-3, ADAM9, ADAMTS1, ADAMTS13, Cathepsin L, BILE ACID, Casp_1, Casp_3, Casp_8, Cathepsin A, Cathepsin B, Cathepsin D, Cathepsin S, CCL20, CCL28, CCL5, CX3CL1, CXCL16, DNA, DPPIV, Endoglin, Endostatin/Collagen XVIII, FFA, FGF basic, HGF, HMGB1, HSP70, HSP90, ICAM-1, IGFBP-1, IGFBP-2, IGFBP-3, IL10, IL17A, IL18, IL18BP, IL1a, IL1beta, IL6, IL8, IP10, Kallikrein 6 (KLK6), LDH, Leptin, LiveMitoc, LPS, MCP1, MMP-2, MMP-9, Nucleosomas, PCSK9, Pentraxin 3 (PTX3), Platelet Factor 4 (PF4), Prolactin, Prot, PRTN3, Myeloblastin, PSELECTINE, Serpin E1, Serpin F1, SPECKS, THP1_IL1, TIMP-1, TIMP-4, uPAr, URIC ACID, VCAM-1, VEGF, Cathepsin X, GDNF, Cathepsin Z and/or Cathepsin P, or any combination thereof, in the organ preservation solution or in a blood sample obtained from the patient, and b) wherein the identification of a deviation or variation (increase or decrease) in the concentration or expression level with respect to a pre-established threshold level determined in control subjects, is an indication that the subject may suffer organ transplant rejection.

In a preferred embodiment, the method of the invention refers to an in vitro method for predicting organ transplant rejection in a subject in need thereof which comprises: a) assessing the concentration or expression level of at least a biomarker selected from the group consisting of: Fibronectin, Hyaluronic acid, MIP1B, mitDNA, MMP7, MMP8, Angiogenin and/or ANGPT2, or any combination thereof, in the organ preservation solution or in a blood sample obtained from the patient, and b) wherein the identification of a deviation or variation (increase or decrease) in the concentration or expression level with respect to a pre-established threshold level determined in control subjects, is an indication that the subject may suffer organ transplant rejection.

In a preferred embodiment, the method of the invention refers to an in vitro method for predicting organ transplant rejection in a subject in need thereof which comprises: a) assessing the concentration or expression level of at least a combination of biomarkers selected from Table 8 or Table 9, in the organ preservation solution or in a blood sample obtained from the patient, and b) wherein the identification of a deviation or variation (increase or decrease) in the concentration or expression level with respect to a pre-established threshold level determined in control subjects, is an indication that the subject may suffer organ transplant rejection.

In a preferred embodiment, the method of the invention refers to an in vitro method for predicting organ transplant rejection in a subject in need thereof which comprises assessing the concentration or expression level of the following combination of biomarkers: Fibronectin, Hyaluronic acid, MIP1B, mitDNA, MMP7, MMP8, Angiogenin and ANGPT2, in the organ preservation solution or in a blood sample obtained from the patient, and b) wherein the identification of a deviation or variation (increase or decrease) in the concentration or expression level with respect to a pre-established threshold level determined in control subjects, is an indication that the subject may suffer organ transplant rejection.

In a preferred embodiment, the method of the invention refers to an in vitro method for predicting acute organ transplant rejection, most preferably acute liver transplant rejection, in a subject in need thereof.

The second embodiment of the present invention refers to the in vitro use of at least a biomarker selected from the group consisting of: Fibronectin, Hyaluronic, MIP1B, HLADR_FCM, mitDNA, MMP-7, MMP-8, Angiogenin, Angiopoietin-2, Thrombospondin-1, MMP-3, ADAM9, ADAMTS1, ADAMTS13, Cathepsin L, BILE ACID, Casp_1, Casp_3, Casp_8, Cathepsin A, Cathepsin B, Cathepsin D, Cathepsin S, CCL20, CCL28, CCL5, CX3CL1, CXCL16, DNA, DPPIV, Endoglin, Endostatin/Collagen XVIII, FFA, FGF basic, HGF, HMGB1, HSP70, HSP90, ICAM-1, IGFBP-1, IGFBP-2, IGFBP-3, IL10, IL17A, IL18, IL18BP, IL1a, IL1beta, IL6, IL8, IP10, Kallikrein 6 (KLK6), LDH, Leptin, LiveMitoc, LPS, MCP1, MMP-2, MMP-9, Nucleosomas, PCSK9, Pentraxin 3 (PTX3), Platelet Factor 4 (PF4), Prolactin, Prot, PRTN3, Myeloblastin, PSELECTINE, Serpin E1, Serpin F1, SPECKS, THP1_IL1, TIMP-1, TIMP-4, uPAr, URIC ACID, VCAM-1, VEGF, Cathepsin X, GDNF, Cathepsin Z and/or Cathepsin P, or any combination thereof; or of at least a biomarker selected from the group consisting of: Fibronectin, Hyaluronic acid, MIP1B, mitDNA, MMP7, MMP8, Angiogenin and/or ANGPT2, or any combination thereof, or of at least a combination of biomarker selected from Table 8 or Table 9; or of the following combination of biomarkers: Fibronectin, Hyaluronic acid, MIP1B, mitDNA, MMP7, MMP8, Angiogenin and ANGPT2, for predicting organ transplant rejection in a subject in need thereof.

In a preferred embodiment, the present invention refers to the in vitro use of the above-described biomarkers for predicting acute organ transplant rejection, most preferably acute liver transplant rejection, in a subject in need thereof.

The third embodiment of the present invention refers to a kit for performing the method of the invention which comprises reagents or tools for assessing the concentration or expression level of at least a biomarker selected from the group consisting of: Fibronectin, Hyaluronic, MIP1B, HLADR_FCM, mitDNA, MMP-7, MMP-8, Angiogenin, Angiopoietin-2, Thrombospondin-1, MMP-3, ADAM9, ADAMTS1, ADAMTS13, Cathepsin L, BILE ACID, Casp_1, Casp_3, Casp 8, Cathepsin A, Cathepsin B, Cathepsin D, Cathepsin S, CCL20, CCL28, CCL5, CX3CL1, CXCL16, DNA, DPPIV, Endoglin, Endostatin/Collagen XVIII, FFA, FGF basic, HGF, HMGB1, HSP70, HSP90, ICAM-1, IGFBP-1, IGFBP-2, IGFBP-3, IL10, IL17A, IL18, IL18BP, IL1a, IL1beta, IL6, IL8, IP10, Kallikrein 6 (KLK6), LDH, Leptin, LiveMitoc, LPS, MCP1, MMP-2, MMP-9, Nucleosomas, PCSK9, Pentraxin 3 (PTX3), Platelet Factor 4 (PF4), Prolactin, Prot, PRTN3, Myeloblastin, PSELECTINE, Serpin E1, Serpin F1, SPECKS, THP1_IL1, Thrombospondin-1, TIMP-1, TIMP-4, uPAr, URIC ACID, VCAM-1, VEGF, Cathepsin X, GDNF, Cathepsin Z and/or Cathepsin P, or any combination thereof, or of at least a biomarker selected from the group consisting of: Fibronectin, Hyaluronic acid, MIP1B, mitDNA, MMP7, MMP8, Angiogenin and/or ANGPT2, or any combination thereof, or of at least a combination of biomarker selected from Table 8 or Table 9; or of the following combination of biomarkers: Fibronectin, Hyaluronic acid, MIP1B, mitDNA, MMP7, MMP8, Angiogenin and ANGPT2.

The fourth embodiment of the present invention refers to the use of the kit for predicting organ transplant rejection, preferably for predicting liver transplant rejection.

Additionally, the present invention refers to:

An in vitro method for identifying biomarker signatures for predicting organ transplant rejection, preferably for predicting liver transplant rejection, which comprises: a) Assessing the concentration or expression level of at least one of the above cited biomarkers, or any combination thereof, in the organ preservation solution or in a blood sample obtained from the patient, and b) wherein the identification of a deviation or variation (increase or decrease) in the concentration or expression level with respect to a pre-established threshold level determined in control subjects, is an indication that the biomarker signature can be used for predicting organ transplant rejection.

A method for detecting any of the above cited biomarkers, or combinations thereof, in the organ preservation solution or in a blood sample obtained from the patient at risk of suffering organ transplant rejection, the method, comprising:
a) contacting the test sample with a primer or antibody specific to the biomarkers.
b) amplifying the biomarker in the case of using a primer to produce an amplification product in the test sample or amplify the signal by using a second antibody in the case of using and antibody for detection; and
c) measuring the expression level by determining the level of the amplification product or signal in the test sample.

The present invention also refers to a method for treating a patient who has been identified as probably suffering from organ transplant rejection by using the method of the invention, wherein the treatment comprises immunosuppressants such as: Cyclosporines (Neoral®, Gengraf®, Sandimmune®), Tacrolimus (Prograf®, FK506), Mycophenolate mofetil (CellCept®), Prednisone, Azathioprine (Imuran®), Sirolimus (Rapamune®), Daclizumab and Basiliximab (Zenapax® and Simulect®), OKT3@(monoclonal antibody), Thymoglobulin and/or Campath.

In a preferred embodiment, the method of the invention is a computer-implemented invention, wherein a processing unit (hardware) and a software are configured to:
Receive the concentration or expression values of any of the above cited biomarkers or combinations thereof,
Process the concentration or expression values received for finding substantial variations or deviations, and
Provide an output through a terminal display of the variation or deviation of the concentration or expression level, wherein the variation or deviation of the concentration or expression level indicates that the subject may suffer from organ transplant rejection.

The present invention also refers to a computer program or a computer-readable media containing means for carrying out a method of the invention.

For the purpose of the present invention the following terms are defined:
The term “comprising” means including, but not limited to, whatever follows the word “comprising”. Thus, use of the term “comprising” indicates that the listed elements are required or mandatory, but that other elements are optional and may or may not be present.
The term “consisting of” means including, and limited to, whatever follows the phrase “consisting of”. Thus, the phrase “consisting of” indicates that the listed elements are required or mandatory, and that no other elements may be present.
Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value must be determined in order to obtain the optimal sensitivity and specificity according to the function of the test and the benefit/risk balance (clinical consequences of false positive and false negative). In one embodiment of the present invention, the threshold value is derived from patients not suffering from organ transplant rejection. Typically, the optimal sensitivity and specificity (and so the threshold value) can be determined using a Receiver Operating Characteristic (ROC) curve based on experimental data.

### DESCRIPTION OF THE FIGURES

FIG. 1. Differences between each marker when comparing the non-rejection group (normal; n=47) and the acute rejection group (AR; n=20). Dots represent the median value for each of the markers.

FIG. 2. Heat map of sample classification when using selected markers. A. The heat map represents the clustering established when using different algorithmic measurements. When the color is darker, it indicates a closer affinity to the non-rejection group, while if the color is lighter, it represents a stronger affinity to the rejection group. Two clusters are observed corresponding to the two existing groups. B. Representation of the observed distances between the selected markers when processing the data using 2 algorithmic methodologies. Both groups (Non-rejecters and Rejecters) are well-defined, where colors closer to black indicate shorter distances, meaning patients belonging to the same group, while lighter colors represent greater distances or patients from different groups after compared every patient with the others. A clear clustering was found among all patients who are non-rejecters and patients who are rejecters. (NR: non-rejecters; P: rejecters).

FIG. 3. Graphical representation of the rejection vs non-rejection groups after processing the results with different algorithms. The ellipses represent the dispersion of results for all patients included. There are significant differences between the two groups with a p<0.05.

FIG. 4. A. ROC curve from the data obtained in the rejection and non-rejection groups when the markers are analyzed by using an algorithm comprising the following combination of biomarkers: Fibronectin, Hyaluronic acid, MIP1B, mitDNA, MMP7, MMP8, Angiogenin and ANGPT2. The ROC space was calculated in 99.6%. B. Table showing the calculation of the positive and negative predictive values of both groups (rejection vs non-rejection) when the markers are processed by the algorithm.

### DETAILED DESCRIPTION OF THE INVENTION

The present invention is illustrated by means of the examples below without the intention of limiting its scope of protection.

### Example 1. Material and Methods

### Example 1.1. Determination of Cytokines, Chemokines and Other Soluble Molecules by Enzyme-Linked Immunosorbent Assay (ELISA)

Samples were subjected to ELISA assay by using specific commercial kits for each molecule and following the manufacturer's instructions.

### Example 1.2. Determination of Mitochondrial DNA

Mitochondrial DNA was isolated using a DNeasy Blood &Tissue Kit (QIAGEN). The standard human mitochondrial DNA was obtained from mitochondria isolated. The standard curve of mitochondrial DNA was obtained in each assay for absolute quantification of mitochondrial DNA. Quantitative PCR was employed to measure mitochondrial DNA using TB Green™ Premix Ex Taq (TAKARA BIO INC, Japan) in an iQ5 Real-Time PCR System (Bio-Rad). The following primer was used: human mitochondrial cytochrome b (F: 5h-CCCCACAAACCCCATTACTAAACCCA-3′ (SEQ ID NO: 1) and R 5′-TTCATCATGCGGAGATGTTGGATGG-3′) (SEQ ID NO: 2).

### Example 1.3. Determination of Membrane Protein Expression in Human Umbilical Vein Endothelial Cells (HUVECs)

HUVECs were cultured in Gibco™ Medium 2000 supplemented with Low Serum Growth Supplement (ThermoFisher Scientific) in the absence of antibiotics. 10⁵ cells/well were cultured in 24-well plates and incubated 16 h in the presence of organ preservation solution recollected from explanted livers after cold ischemia storage.

Membrane expression of those molecules was determined by flow cytometry. Cultured HUVECs were detached from wells and then stained with specific fluorescent stained antibodies.

After staining, the cells were subjected to flow cytometry analysis in a BD FACSCanto flow cytometer and FACSDiva software (BD Biosciences) by gating for singlets based on the forward scatter (FSC-A, FSC-H) and side scatter (SSC-A) parameters. Data were analyzed by FCS Express 5 software (DeNovo Software, Pasadena, USA).

### Example 2. Results

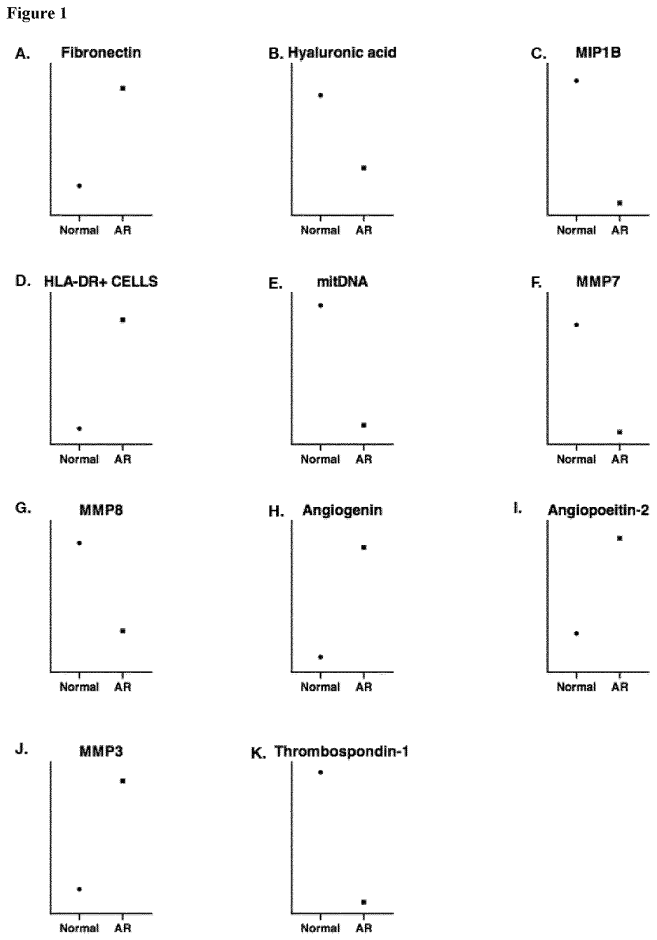

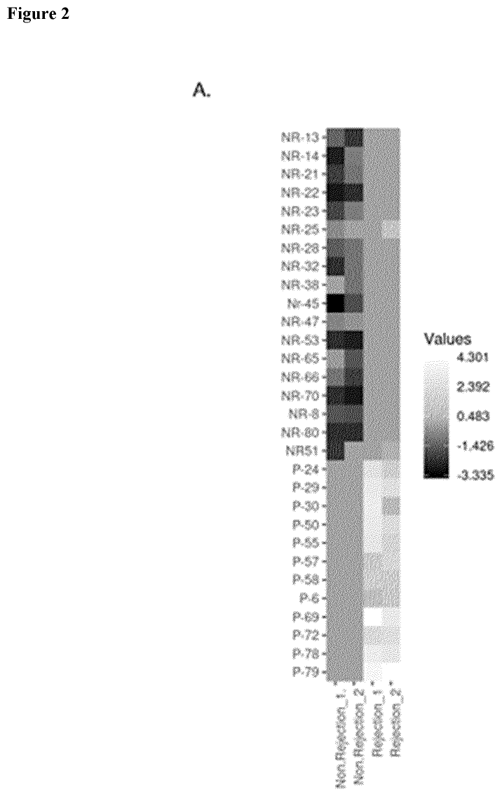

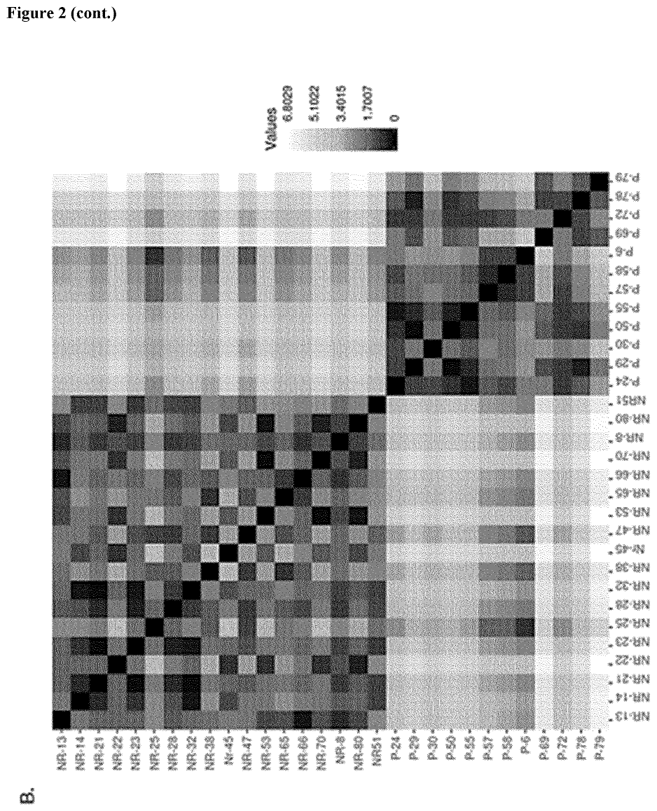

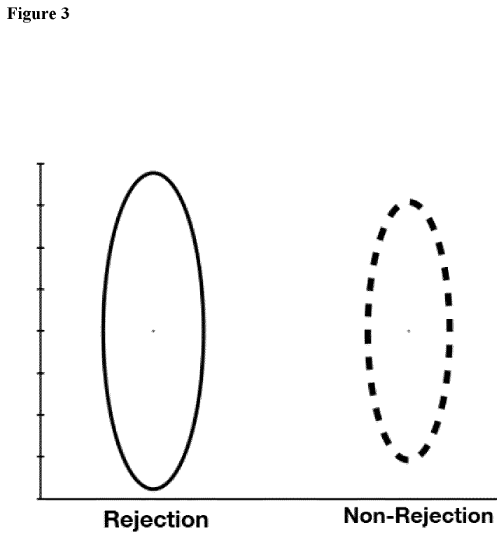

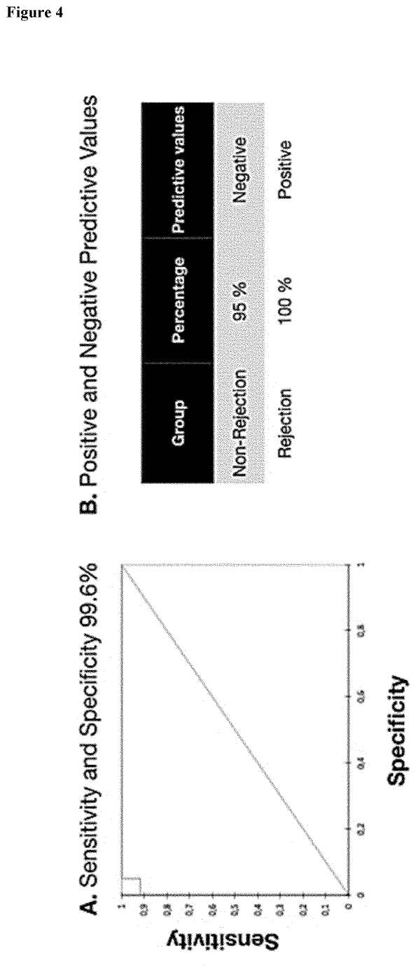

In a prospective clinical study performed in 80 transplanted patients (Table 3 and Table 4), 80 markers (Tables 1 and 2) released by the donor liver have been identified, which are differentially expressed to a greater or lesser degree in those who suffer acute rejection (Table 5, Table 6 and FIG. 1). The detection of these markers is performed in the first 48 hours after liver transplantation. Among all these markers, those with statistical differences of over 0.001 (Table 1) were selected for further analysis. FIGS. 1 to 4 show the existing differences in the levels of the markers whose differences are below 0.001. In turn, the expression levels of the markers were combined with other information to generate an algorithm which comprises the following combination of biomarkers: Fibronectin, Hyaluronic acid, MIP1B, mitDNA, MMP7, MMP8, Angiogenin and ANGPT2 (FIG. 4). Additionally, all other possible combinations were calculated among the markers that showed highly significant differences below 0.001 (Table 8). These highly significant markers were also combined with the remaining markers (Table 9), establishing combinations ranging from 2 markers, 3 markers, and so forth, up to combinations using the total number of markers. The area under the ROC curve was calculated for each marker combination, considering only those combinations that exhibited areas under the curve exceeding 0.7. With these markers, an algorithm was generated and refined until two differentiated groups were obtained (Table 7, FIG. 2 and FIG. 3). This algorithm makes it possible to determine in transplanted patients those who are going to suffer an episode of acute rejection. This algorithm has been validated with dozens of liver transplant patients and shows specificity and sensitivity values of 99.6% (FIG. 4). In addition, the algorithm has positive predictive values of 100%, while the negative predictive values are 95%.

TABLE 1
List of main biomarkers, including the biological process, as well as their identifier
in protein databases (Uniprot ID) and their gene identifier (NCBI Entrez Gene ID).

| | | | NCBI Entrez |
|---|---|---|---|
| Main Biomarker | Biological Process | Uniprot ID | Gene ID |
| Fibronectin; FN | Extracellular Matrix, cell adhesion, | P02751 | 2335 |
| | Angiogenesis, endothelial cell | | |
| | differentiation, acute-phase response, | | |
| | peptide cross linking and secretion | | |
| Hyaluronan; | Extracellular Matrix, cell matrix | Not Uniprot ID or Gene because | |
| Hyaluronic acid | adhesion, anatomical structure | it is a glycosaminoglycan | |
| | morphogenesis, mitochondria, catabolic | | |
| | processes, Angiogenesis, Regulation of | | |
| | cellular extravasation, Response to | | |
| | Wound | | |
| MIP1B; Macrophage | Acute-phase response, cell matrix | P13236 | 6351 |
| Inflammatory Protein | adhesion | | |
| 1-Beta | cell-cell signaling and signal transduction | | |
| | immune response | | |
| | inflammatory response | | |
| | Inmune cell chemotaxis | | |
| | Regulation of calcium | | |
| | Regulation of ERK1 and ERK2 cascade | | |
| | Regulation of GTPase activity, | | |
| | mitochondria | | |
| | Response to toxic insults | | |
| HLADR_FCM; | Antigen processing and presentation, | Not Uniprot ID or Gene because | |
| Expression of | inflammation, immunity, DNA damage | it is a lab procedure | |
| membrane HLA-DR in | responses, Gene expression, MHC class | | |
| HUVEC cells | II, Apoptosis, Cell migration, signaling | | |
| | and differentiation; peptide cross linking | | |
| | and secretion, Regulation of ERK1 and | | |
| | ERK2 cascade | | |
| mitDNA; | mitochondria, gene expression, catabolic | Not Uniprot ID or Gene because | |
| mithochondrial DNA | processes | it is a DNA | |
| MMP7; Matrix | Extracellular Matrix, Proteolysis, cell | P09237 | 4316 |
| Metalloproteinase-7; | migration, catabolic processes | | |
| Matrilysin | | | |
| MMP8; Matrix | Extracellular Matrix, Proteolysis, cell | P2289 | 4317 |
| Metalloproteinase-8; | migration, Gene Expression, | | |
| Neutrophil Collagenase | Inflammation, Immunity, Angiogenesis, | | |
| | Mitochondria, Apoptosis. | | |
| Angiogenin | Angiogenesis, Inmunity, Inflammation, | P03950 | 283 |
| | cell migration and communication, | | |
| | endothelial cell proliferation, protein | | |
| | secretion, Protein expression, catabolic | | |
| | processes. | | |
| ANGPT2; | Angiogenesis, organ morphogenesis and | O15123 | 285 |
| Angiopoietin-2 | regeneration, gene expression, | | |
| | endothelial cell migration, cell adhesion, | | |
| | chemotaxis, metabolism, signal | | |
| | transduction | | |
| THBS1; | Cell adhesion and migration, organ | P07996 | 7057 |
| Thrombospondin-1 | morphogenesis and regeneration, | | |
| | Inflammation, apoptosis, angiogenesis, | | |
| | immunity, endothelial cell migration and | | |
| | proliferation, cell matrix adhesion, | | |
| MMP3; Matrix | mitochondria, catabolic processes, ECM, | P08254 | 4314 |
| Metalloproteinase-3; | apoptosis, proteolysis, inflammation. | | |
| Stromelysin-1 | | | |

TABLE 2
List of other biomarkers, including the biological process, as well as their identifier
in protein databases (Uniprot ID) and their gene identifier (NCBI Entrez Gene ID).

| Other biomarker | Complete name | Biological Process |
|---|---|---|
| ADAM9 | Disintegrin And | Catabolic processes; Cell differentiation, |
| | Metalloproteinase Domain- | proliferation, migration, communication and |
| | Containing Protein 9 | extravasation; Cell matrix adhesion and |
| | | chemotaxis; Immunity; Inflammation and acute- |
| | | phase responses; Mitochondria and |
| | | metabolism; Protein and peptide Expression, |
| | | Secretion and Cross-linking, stability and |
| | | localization; Proteolysis; Regulation of ERK1 and |
| | | ERK2 cascade and calcium; |
| ADAMTS1 | ADAM Metallopeptidase | Angiogenesis; Cell differentiation, proliferation, |
| | With Thrombospondin Type | migration, communication and extravasation; |
| | 1 Motif 1 | cell-cell signaling and signal transduction; |
| | | Extracellular Matrix; Gene expression and DNA |
| | | damage responses; |
| ADAMTS13 | ADAM Metallopeptidase | Cell matrix adhesion and chemotaxis; cell-cell |
| | With Thrombospondin Type | signaling and signal transduction; Extracellular |
| | 1 Motif 13 | Matrix; Inflammation and acute-phase |
| | | responses; Mitochondria and metabolism; |
| | | Protein and peptide Expression, Secretion and |
| | | Cross-linking, stability and localization; |
| | | Proteolysis; Response to toxic insults; |
| Cathepsin L | Cathepsin L | Catabolic processes; Immunity; Protein and |
| | | peptide Expression, Secretion and Cross-linking, |
| | | stability and localization; Proteolysis; |
| BILE ACID | Bile acid | Digestion Process; Liver Damage |
| Casp_1 | Caspase 1 | Apoptosis and cell death; Proteolysis; Protein |
| | | and peptide Expression, Secretion and Cross- |
| | | linking, stability and localization; Inflammation |
| | | and acute-phase responses; Immunity; |
| Casp_3 | Caspase 3 | Angiogenesis; Apoptosis and cell death; Cell |
| | | differentiation, proliferation, migration, |
| | | communication and extravasation; Cell matrix |
| | | adhesion and chemotaxis; Gene expression and |
| | | DNA damage responses; Proteolysis; Protein |
| | | and peptide Expression, Secretion and Cross- |
| | | linking, stability and localization; Immunity; |
| Casp_8 | Caspase 8 | Angiogenesis; Apoptosis and cell death; Cell |
| | | differentiation, proliferation, migration, |
| | | communication and extravasation; Cell matrix |
| | | adhesion and chemotaxis; Regulation of ERK1 |
| | | and ERK2 cascade and calcium; Mitochondria |
| | | and metabolism; Organ and liver anatomical |
| | | structure morphogenesis and regeneration; |
| Cathepsin A | Cathepsin A | Apoptosis and cell death; Proteolysis; Protein |
| | | and peptide Expression, Secretion and Cross- |
| | | linking, stability and localization; |
| Cathepsin B | Cathepsin B | Catabolic processes; Cell differentiation, |
| | | proliferation, migration, communication and |
| | | extravasation; Proteolysis; Protein and peptide |
| | | Expression, Secretion and Cross-linking, stability |
| | | and localization; |
| Cathepsin D | Cathepsin D | Apoptosis and cell death; Catabolic processes; |
| | | Proteolysis; Protein and peptide Expression, |
| | | Secretion and Cross-linking, stability and |
| | | localization; Immunity; |
| Cathepsin S | Cathepsin S | Catabolic processes; Extracellular Matrix; |
| | | Proteolysis; Protein and peptide Expression, |
| | | Secretion and Cross-linking, stability and |
| | | localization; Immunity; |
| CCL20 | C-C Motif Chemokine Ligand | Cell matrix adhesion and chemotaxis; cell-cell |
| | 20 | signaling and signal transduction; Regulation of |
| | | ERK1 and ERK2 cascade and calcium; |
| | | Inflammation and acute-phase responses; |
| | | Immunity; |
| CCL28 | C-C Motif Chemokine Ligand | Cell matrix adhesion and chemotaxis; |
| | 28 | Regulation of ERK1 and ERK2 cascade and |
| | | calcium; Mitochondria and metabolism; |
| | | Immunity; |
| CCL5 | C-C Motif Chemokine Ligand | Catabolic processes; Cell matrix adhesion and |
| | 6 | chemotaxis; cell-cell signaling and signal |
| | | transduction; Regulation of GTPase activity; |
| | | Regulation of ERK1 and ERK2 cascade and |
| | | calcium; Inflammation and acute-phase |
| | | responses; |
| CX3CL1 | C-X3-C Motif Chemokine | Aging; Angiogenesis; Cell differentiation, |
| | Ligand 1 | proliferation, migration, communication and |
| | | extravasation; Cell matrix adhesion and |
| | | chemotaxis; cell-cell signaling and signal |
| | | transduction; Response to Wound; |
| | | Inflammation and acute-phase responses; |
| | | Immunity; |
| CXCL16 | C-X-C Motif Chemokine | Aging; Angiogenesis; Cell differentiation, |
| | Ligand 16 | proliferation, migration, communication and |
| | | extravasation; Cell matrix adhesion and |
| | | chemotaxis; cell-cell signaling and signal |
| | | transduction; Response to Wound; |
| | | Inflammation and acute-phase responses; |
| | | Immunity; |
| DNA | Deoxyribonucleic acid | Cellular Death; Cellular and Tissue Damage |
| DPPIV; DPPIV_CD26 | Dipeptidyl Peptidase 4 | Protein and peptide Expression, Secretion and |
| | | Cross-linking, stability and localization; |
| Endoglin | Endoglin | Angiogenesis; Cell differentiation, proliferation, |
| | | migration, communication and extravasation; |
| | | Cell matrix adhesion and chemotaxis; cell-cell |
| | | signaling and signal transduction; Extracellular |
| | | Matrix; Response to Wound; Mitochondria and |
| | | metabolism; Organ and liver anatomical |
| | | structure morphogenesis and regeneration; |
| | | Protein and peptide Expression, Secretion and |
| | | Cross-linking, stability and localization; |
| Endostatin/ | Endostatin/ | Angiogenesis; Cell matrix adhesion and |
| Collagen XVIII | Collagen XVIII | chemotaxis; cell-cell signaling and signal |
| | | transduction; Endothelial cell differentiation, |
| | | proliferation and migration; Extracellular Matrix; |
| FFA | Free fatty acid | Cell differentiation, proliferation, migration, |
| | | communication and extravasation; |
| | | Mitochondria and metabolism; Protein and |
| | | peptide Expression, Secretion and Cross-linking, |
| | | stability and localization; Inflammation and |
| | | acute-phase responses; Immunity; |
| FGF basic | Basic Fibroblast Growth | Aging; Angiogenesis; Cell differentiation, |
| | Factor | proliferation, migration, communication and |
| | | extravasation; Cell matrix adhesion and |
| | | chemotaxis; Endothelial cell differentiation, |
| | | proliferation and migration; Gene expression |
| | | and DNA damage responses; Response to |
| | | Wound; Regulation of ERK1 and ERK2 cascade |
| | | and calcium; Organ and liver anatomical |
| | | structure morphogenesis and regeneration; |
| HGF | Hepatocyte Growth Factor | Angiogenesis; Apoptosis and cell death; Cell |
| | | matrix adhesion and chemotaxis; cell-cell |
| | | signaling and signal transduction; Endothelial |
| | | cell differentiation, proliferation and migration; |
| | | Organ and liver anatomical structure |
| | | morphogenesis and regeneration; |
| HMGB1 | High Mobility Group Box 1 | Apoptosis and cell death; Endothelial cell |
| | | differentiation, proliferation and migration; |
| | | Gene expression and DNA damage responses; |
| | | Regulation of ERK1 and ERK2 cascade and |
| | | calcium; Protein and peptide Expression, |
| | | Secretion and Cross-linking, stability and |
| | | localization; Inflammation and acute-phase |
| | | responses; Immunity; |
| HSP70 | Heat shock protein 70 | Protein and peptide Expression, Secretion and |
| | | Cross-linking, stability and localization; |
| HSP90 | Heat shock protein 90 | Apoptosis and cell death; Mitochondria and |
| | | metabolism; Protein and peptide Expression, |
| | | Secretion and Cross-linking, stability and |
| | | localization; |
| ICAM-1 | Intercellular Adhesion | Apoptosis and cell death; Cell matrix adhesion |
| | Molecule 1 | and chemotaxis; cell-cell signaling and signal |
| | | transduction; Endothelial cell differentiation, |
| | | proliferation and migration; Regulation of ERK1 |
| | | and ERK2 cascade and calcium; Inflammation |
| | | and acute-phase responses; Immunity; |
| IGFBP-1 | Insulin Like Growth Factor | Aging; Cell differentiation, proliferation, |
| | Binding Protein 1 | migration, communication and |
| | | extravasation; cell-cell signaling and signal |
| | | transduction; Organ and liver anatomical |
| | | structure morphogenesis and regeneration; |
| | | Inflammation and acute-phase responses; |
| IGFBP-2 | Insulin Like Growth Factor | Aging; Cell differentiation, proliferation, |
| | Binding Protein 2 | migration, communication and extravasation; |
| | | cell-cell signaling and signal transduction; |
| | | Mitochondria and metabolism; Organ and liver |
| | | anatomical structure morphogenesis and |
| | | regeneration; Immunity; |
| IGFBP-3 | Insulin Like Growth Factor | Apoptosis and cell death; Cell differentiation, |
| | Binding Protein 3 | proliferation, migration, communication and |
| | | extravasation; cell-cell signaling and signal |
| | | transduction; Protein and peptide Expression, |
| | | Secretion and Cross-linking, stability and |
| | | localization; |
| IL10 | interleukin 10 | Aging; Apoptosis and cell death; Cell matrix |
| | | adhesion and chemotaxis; Endothelial cell |
| | | differentiation, proliferation and migration; |
| | | Response to Wound; Mitochondria and |
| | | metabolism; Organ and liver anatomical |
| | | structure morphogenesis and regeneration; |
| | | Inflammation and acute-phase responses; |
| | | Immunity; |
| IL17A | interleukin 17a | Apoptosis and cell death; cell-cell signaling and |
| | | signal transduction; Gene expression and DNA |
| | | damage responses; Inflammation and acute- |
| | | phase responses; Immunity; |
| IL18 | interleukin 18 | Angiogenesis; Cell differentiation, proliferation, |
| | | migration, communication and extravasation; |
| | | cell-cell signaling and signal transduction; Gene |
| | | expression and DNA damage responses; Protein |
| | | and peptide Expression, Secretion and Cross- |
| | | linking, stability and localization; Inflammation |
| | | and acute-phase responses; |
| IL18BP | interleukin 18 binding | Angiogenesis; Cell differentiation, proliferation, |
| | protein | migration, communication and extravasation; |
| | | cell-cell signaling and signal transduction; Gene |
| | | expression and DNA damage responses; Protein |
| | | and peptide Expression, Secretion and Cross- |
| | | linking, stability and localization; Inflammation |
| | | and acute-phase responses; |
| IL1a | interleukin 1 alpha | Apoptosis and cell death; Cell |
| | | differentiation, proliferation, migration, |
| | | communication and extravasation; Gene |
| | | expression and DNA damage responses; |
| | | Response to Wound; Regulation of ERK1 |
| | | and ERK2 cascade and calcium; |
| | | Mitochondria and metabolism; Organ and |
| | | liver anatomical structure morphogenesis |
| | | and regeneration; Protein and peptide |
| | | Expression, Secretion and Cross-linking, |
| | | stability and localization; Inflammation and |
| | | acute-phase responses; Immunity; |
| IL1beta | interleukin 1 beta | Apoptosis and cell death; Catabolic processes; |
| | | Cell differentiation, proliferation, migration, |
| | | communication and extravasation; Cell matrix |
| | | adhesion and chemotaxis; cell-cell signaling and |
| | | signal transduction; Endothelial cell |
| | | differentiation, proliferation and migration; |
| | | Gene expression and DNA damage responses; |
| | | Regulation of ERK1 and ERK2 cascade and |
| | | calcium; Organ and liver anatomical structure |
| | | morphogenesis and regeneration; Protein and |
| | | peptide Expression, Secretion and Cross-linking, |
| | | stability and localization; Inflammation and |
| | | acute-phase responses; Immunity; |
| IL6 | interleukin 6 | Angiogenesis; Apoptosis and cell death; Cell |
| | | differentiation, proliferation, migration, |
| | | communication and extravasation; Gene |
| | | expression and DNA damage responses; |
| | | Mitochondria and metabolism; Organ and liver |
| | | anatomical structure morphogenesis and |
| | | regeneration; Inflammation and acute-phase |
| | | responses; Immunity; |
| IL8 | interleukin 8 | Angiogenesis; Cell matrix adhesion and |
| | | chemotaxis; cell-cell signaling and signal |
| | | transduction; Gene expression and DNA |
| | | damage responses; Regulation of ERK1 and |
| | | ERK2 cascade and calcium; Inflammation and |
| | | acute-phase responses; Immunity; |
| IP10 | Interferon-Inducible | Angiogenesis; Cell differentiation, proliferation, |
| | Cytokine IP-10 | migration, communication and extravasation; |
| | | Cell matrix adhesion and chemotaxis; cell-cell |
| | | signaling and signal transduction; Endothelial |
| | | cell differentiation, proliferation and migration; |
| | | Gene expression and DNA damage responses; |
| | | Mitochondria and metabolism; Organ and liver |
| | | anatomical structure morphogenesis and |
| | | regeneration; Protein and peptide Expression, |
| | | Secretion and Cross-linking, stability and |
| | | localization; Inflammation and acute-phase |
| | | responses; Immunity; |
| Kallikrein 6, KLK6 | Kallikrein 6 | Catabolic processes; Cell differentiation, |
| | | proliferation, migration, communication and |
| | | extravasation; Extracellular Matrix; Response to |
| | | Wound; Proteolysis; Mitochondria and |
| | | metabolism; Organ and liver anatomical |
| | | structure morphogenesis and regeneration; |
| | | Protein and peptide Expression, Secretion and |
| | | Cross-linking, stability and localization; |
| LDH | Lactate dehydrogenase | Mitochondria and metabolism; Inflammation |
| | | and acute-phase responses; Angiogenesis; |
| Leptin | Leptin | Angiogenesis; Apoptosis and cell death; Cell |
| | | differentiation, proliferation, migration, |
| | | communication and extravasation; cell-cell |
| | | signaling and signal transduction; Response to |
| | | Wound; Mitochondria and metabolism; Organ |
| | | and liver anatomical structure morphogenesis |
| | | and regeneration; Protein and peptide |
| | | Expression, Secretion and Cross-linking, stability |
| | | and localization; Inflammation and acute-phase |
| | | responses; Immunity; |
| LiveMitoc | Complete free mitochondria | Cellular Death; Cellular and Tissue Damage |
| LPS | Lypopolisaccharide | Inflammation and acute-phase responses; |
| MCP1 | Monocyte Chemoattractant | Angiogenesis; Cell differentiation, proliferation, |
| | Protein-1 | migration, communication and extravasation; |
| | | Cell matrix adhesion and chemotaxis; cell-cell |
| | | signaling and signal transduction; Endothelial |
| | | cell differentiation, proliferation and migration; |
| | | Regulation of GTPase activity; Regulation of |
| | | ERK1 and ERK2 cascade and calcium; |
| | | Mitochondria and metabolism; Organ and liver |
| | | anatomical structure morphogenesis and |
| | | regeneration; Inflammation and acute-phase |
| | | responses; Immunity; |
| MMP-2 | Matrix Metalloproteinase 2 | Aging; Angiogenesis; Apoptosis and cell death; |
| | | Catabolic processes; Cell differentiation, |
| | | proliferation, migration, communication and |
| | | extravasation; Cell matrix adhesion and |
| | | chemotaxis; Endothelial cell differentiation, |
| | | proliferation and migration; Extracellular Matrix; |
| | | Response to Wound; Mitochondria and |
| | | metabolism; Organ and liver anatomical |
| | | structure morphogenesis and regeneration; |
| | | Inflammation and acute-phase responses; |
| MMP-9 | Matrix Metalloproteinase 3 | Apoptosis and cell death; Catabolic processes; |
| | | Cell differentiation, proliferation, migration, |
| | | communication and extravasation; Extracellular |
| | | Matrix; Proteolysis; Regulation of ERK1 and |
| | | ERK2 cascade and calcium; Mitochondria and |
| | | metabolism; Organ and liver anatomical |
| | | structure morphogenesis and regeneration; |
| | | Inflammation and acute-phase responses; |
| | | Immunity; |
| Nucleosomes | Nucleosomes | Cellular Death; Cellular and Tissue Damage |
| PCSK9 | Proprotein Convertase | Apoptosis and cell death; cell-cell signaling and |
| | Subtilisin/Kexin Type 9 | signal transduction; Mitochondria and |
| | | metabolism; Organ and liver anatomical |
| | | structure morphogenesis and regeneration; |
| Pentraxin 3 (PTX3) | Pentraxin 3 | Extracellular Matrix; Inflammation and acute- |
| | | phase responses; Immunity; |
| Platelet Factor | Platelet Factor 4 | Cell differentiation, proliferation, migration, |
| 4 (PF4) | | communication and extravasation; Cell matrix |
| | | adhesion and chemotaxis; Protein and peptide |
| | | Expression, Secretion and Cross-linking, stability |
| | | and localization; Inflammation and acute-phase |
| | | responses; Immunity; |
| Prolactin | Prolactin | Angiogenesis; Cell differentiation, proliferation, |
| | | migration, communication and extravasation; |
| | | cell-cell signaling and signal transduction; |
| | | Mitochondria and metabolism; |
| Prot | Total protein | |
| PRTN3 | Myeloblastin | Cell differentiation, proliferation, migration, |
| | | communication and extravasation; Regulation of |
| | | GTPase activity; Response to Wound; |
| | | Proteolysis; Protein and peptide Expression, |
| | | Secretion and Cross-linking, stability and |
| | | localization; Immunity; |
| PSELECTINE | PSELECTINE | Cell matrix adhesion and chemotaxis; |
| | | Regulation of ERK1 and ERK2 cascade and |
| | | calcium; Inflammation and acute-phase |
| | | responses; Immunity; |
| Serpin E1 | Serpin E1 | Angiogenesis; Cell differentiation, proliferation, |
| | | migration, communication and extravasation; |
| | | Cell matrix adhesion and chemotaxis; |
| | | Inflammation and acute-phase responses; |
| | | Immunity; |
| Serpin F1 | Serpin F1 | Aging; Angiogenesis; Endothelial cell |
| | | differentiation, proliferation and migration; |
| | | Response to toxic insults; Mitochondria and |
| | | metabolism; Organ and liver anatomical |
| | | structure morphogenesis and regeneration; |
| SPECKS | Soluble specks of ASC | Inflammation and acute-phase responses; |
| TIMP-1 | TIMP Metallopeptidase | Aging; Apoptosis and cell death; Cell |
| | Inhibitor 1 | differentiation, proliferation, migration, |
| | | communication and extravasation; Response to |
| | | toxic insults; Response to Wound; Proteolysis; |
| | | Organ and liver anatomical structure |
| | | morphogenesis and regeneration; Inflammation |
| | | and acute-phase responses; |
| TIMP-4 | TIMP Metallopeptidase | Response to toxic insults; Organ and liver |
| | Inhibitor 4 | anatomical structure morphogenesis and |
| | | regeneration; Inflammation and acute-phase |
| | | responses; Immunity; |
| uPAr | Urokinase Plasminogen | Angiogenesis; Apoptosis and cell death; Cell |
| | Activator Surface Receptor | matrix adhesion and chemotaxis; cell-cell |
| | | signaling and signal transduction; Gene |
| | | expression and DNA damage responses; |
| | | Proteolysis; Mitochondria and metabolism; |
| URIC ACID | Uric acid | Hepatic Damage |
| VCAM-1 | Vascular Cell Adhesion | Aging; Cell matrix adhesion and chemotaxis; |
| | Molecule 1 | Response to toxic insults; Regulation of ERK1 |
| | | and ERK2 cascade and calcium; Mitochondria |
| | | and metabolism; Inflammation and acute-phase |
| | | responses; |
| VEGF | Vascular Endothelial Growth | Angiogenesis; Apoptosis and cell death; Cell |
| | Factor | differentiation, proliferation, migration, |
| | | communication and extravasation; Cell matrix |
| | | adhesion and chemotaxis; Endothelial cell |
| | | differentiation, proliferation and migration; |
| | | Gene expression and DNA damage responses; |
| | | Response to Wound; Regulation of ERK1 and |
| | | ERK2 cascade and calcium; Mitochondria and |
| | | metabolism; Organ and liver anatomical |
| | | structure morphogenesis and regeneration; |
| | | Protein and peptide Expression, Secretion and |
| | | Cross-linking, stability and localization; |
| | | Immunity; |
| Cathepsin X | Cathepsin X | Proteolysis; |
| GDNF | Glial Cell Derived | Proteolysis; |
| | Neurotrophic Factor | |
| Cathepsin Z | Cathepsin Z | Proteolysis; Organ and liver anatomical |
| | | structure morphogenesis and regeneration; |
| | | Protein and peptide Expression, Secretion and |
| | | Cross-linking, stability and localization; |
| Cathepsin P | Cathepsin P | Proteolysis; |

TABLE 3
Demographic and clinical data of total organ donors and receptors.
Continuous variables are expressed as mean ± SD; median (range).
Qualitative variables are expressed as frequency (%). DBD, donation
after brain death; DCD, donation after cardiac death; NRP, normothermic
regional perfusion; HCV, hepatitis C virus; HBV, hepatitis B virus;
NASH, non-alcoholic steohepatitis; KT, Kehr tube.

| Variables | Donors (n = 79) | | Receptors (n = 79) | | p |
|---|---|---|---|---|---|
| Age | 60.6 ± 14.2; | | 57.0 ± 9.6; | | 0.126 |
| | 65 (22-87) | | 58.5 (24-72) | | |
| Sex | | | | | |
| Male | 42 | (53) | 44 | (65) | 0.181 |
| Female | 37 | (47) | 24 | (35) | |
| Body mass index | 25.6 ± 3.9; | | 26.8 ± 4.2; | | 0.056 |
| | 25.2 (16.7-36.2) | | 26.7 (18.7-36.8) | | |
| Donation | | | | | |
| DBD | 49 | (62) | | | |
| DCD | 30 | (38) | | | |
| NRP | | | | | |
| Yes | 8 | (27) | | | |
| No | 22 | (73) | | | |

| | | | |
|---|---|---|---|
| Cold Ischemia (min) | 331.8 ± 151.4; | | |
| | 300 (90-960) | | |
| Diseases | | | |
| Alcoholic cirrhosis | | 37 | (46.8) |
| HCV | | 8 | (10.1) |
| Arterial thrombosis | | 6 | (7.6) |
| Primary biliary | | 5 | (6.3) |
| cirrhosis | | 4 | (5.1) |
| Cryptogenic liver | | 4 | (5.1) |
| Cirrhosis | | 3 | (3.8) |
| NASH | | 3 | (3.8) |
| HBV | | 3 | (3.8) |
| Polycystic disease | | 6 | (7.6) |
| Autoimmune hepatitis | | | |
| Other | | | |
| Re-transplant | | 11 | (14) |
| Anastomoses | | | |
| With KT | | 25 | (32) |
| Without KT | | 54 | (68) |
| Exitus at surgery | | 2 | (2.5) |
| Acute rejection | | 20 | (25) |

Continuous variables are expressed as mean ± SD; median (range). Qualitative variables are expressed as frequency (%). DBD, donation after brain death; DCD, donation after cardiac death; NRP, normothermic regional perfusion; HCV, hepatitis C virus; HBV, hepatitis B virus; NASH, non-alcoholic steohepatitis; KT, Kehr tube.

TABLE 4
Demographic and clinical data of organ donors and receptors which
fulfil the inclusion criteria (No DCD, no re-transplant and no anastomoses
with a Kehr tube). Continuous variables are expressed as mean ±
SD; median (range). Qualitative variables are expressed as frequency
HBV, hepatitis B virus; NASH, non-alcoholic steohepatitis.

| Variables | Donors (n = 25) | Receptors (n = 25) | | p |
|---|---|---|---|---|
| Age | 63.9 ± 10.7; | 55.7 ± 12.6; | | 0.017 |
| | 65 (43-87) | 60 (24-71) | | |
| Sex | | | | |
| Male | 10 (40) | 16 | (64) | 0.001 |
| Female | 15 (60) | 9 | (36) | |
| Body mass index | 24.6 ± 4.6; | 25.8 ± 4.0; | | 0.343 |
| | 23.7 (18.4-36.2) | 25.6 (19.5-34.7) | | |
| Cold Ischemia (min) | 285.2 ± 104.8; | | | |
| | 290 (90-543) | | | |
| Diseases | | | | |
| Alcoholic cirrhosis | | 15 | (60) | |
| Primary biliary | | 1 | (4) | |
| cirrhosis | | 1 | (4) | |
| Cryptogenic liver | | 4 | (16) | |
| Cirrhosis | | 2 | (8) | |
| NASH | | 1 | (4) | |
| HBV | | 1 | (4) | |
| Polycystic disease | | | | |
| Other | | | | |
| Acute rejection | | 6 | (24) | |

Continuous variables are expressed as mean ± SD; median (range). Qualitative variables are expressed as frequency HBV, hepatitis B virus: NASH, non-alcoholic steohepatitis.

TABLE 5
Quantification of selected markers in the studied
samples. Variables are expressed as median (range).

| Marker | Concentration | |
|---|---|---|
| Fibronectin (nmol/μl) | 1,592 | (18-17,497) |
| Hyaluronan (nmol/μl) | 80.84 | (12.14-323.40) |
| Mitochondrial DNA (pg/ml) | 5.27 | (0.10-125.70) |
| MIP1B (pg/ml) | 87.45 | (0-629.8) |
| MMP3 (pg/ml) | 232.3 | (15.07-4,649) |
| MMP7 (pg/ml) | 242.4 | (62.07-1,653) |
| MMP8 (pg/ml) | 744.1 | (2.81-9,219) |
| Angiogenin (pg/ml) | 96.45 | (4.94-1,765) |

Variables are expressed as median (range).

TABLE 6
Quantification of the rest of markers in the studied
samples. Variables are expressed as median (range).

| Marker | Concentration | |
|---|---|---|
| Endostatin (pg/ml) | 9,817 | (42.6-137,950) |
| MMP2 (pg/ml) | 396.3 | (78.07-1,800) |
| TIMP1 (pg/ml) | 131,618 | (6,106-463,700) |
| Endoglin (pg/ml) | 641.2 | (135-5,488) |
| uPAR (pg/ml) | 4,063 | (723-27,991) |
| Serpin-F1 (pg/ml) | 294,777 | (75,712-822,371) |
| DPPIV/CD26 (pg/ml) | 8,527 | (753-40,661) |
| IGFBP-3 (pg/ml) | 19,174 | (5,216-79,844) |
| Cathepsin L (pg/ml) | 1.44 | (1.11-19.04) |
| Cathepsin Z (pg/ml) | 62.31 | (45.56-85.86) |
| Uric acid (nmol/μl) | 0.26 | (0.03-0.65) |
| Bile acid (μM) | 55.91 | (0-6,808) |
| Caspase-1 (Relative units/ml) | 37.4 | (0-8,210) |
| Caspase-3 (Relative units/ml) | 38 | (0-5,701) |
| Caspase-8 (Relative units/ml) | 118.8 | (0-34,813) |
| CCL20 (pg/ml) | 75.18 | (0-5,113) |
| CCL28 (pg/ml) | 167.4 | (0-1,057) |
| CCL5 (pg/ml) | 242.5 | (16.61-892.4) |
| CX3CL1 (pg/ml) | 10,418 | (0-2,366,039) |
| CXCL16 (pg/ml) | 1,981 | (295.1-9,057) |
| Nuclear DNA (ng/ml) | 271.3 | (139-242.2) |
| Free fatty acids (nmol/μl) | 0.43 | (0-0.71) |
| HMGB1 (pg/ml) | 120.7 | (28.17-242.2) |
| HSP70 (pg/ml) | 51.2 | (0-1,469) |
| HSP90 (pg/ml) | 83.88 | (0.12-134.2) |
| ICAM-1 (pg/ml) | 152,872 | (22,823-419,780) |
| VCAM-1 (pg/ml) | 11,298 | (2,200-101,176) |
| IL-10 (pg/ml) | 0.25 | (0-15.39) |
| IL-6 (pg/ml) | 62.94 | (0-1,059) |
| IL-8 (pg/ml) | 3.42 | (0-337) |
| IP10 (pg/ml) | 172.1 | (0-995) |
| LDH (Relative units/ml) | 2.5 | (1-4.2) |
| Live mitochondria (number/ml) | 34,764 | (3,013-195,093) |
| LPS (EU/ml) | 0.077 | (0.039-0.242) |
| MIP-1A (pg/ml) | 4 | (0-19.31) |
| MCP1 (pg/ml) | 1,270 | (0-8,863) |
| Nucleosomes (Relative units/ml) | 37 | (6-183) |
| Soluble specks of ASC (number/ml) | 25,762 | (1,936-305,204) |
| P-Selectine (pg/ml) | 67,005 | (0-295,855) |
| IL-18 (pg/ml) | 94.51 | (0-5,998) |
| IL-18BP (pg/ml) | 302.2 | (101.1-1,175) |

Variables are expressed as median (range).

TABLE 7
Confusion Matrix after processing every sample by 2 different
algorithmic methodologies, for all selected biomarkers.
Method 1 samples were clustered in 2 different groups (Rejection
and Non Renection). Method 2, samples were clustered in
3 different categories, rejection, non-rejection and surveillance.
The column labelled as total represents percentage of the
number of algorithm matches when a patient is a rejecter
or non-rejecter for selected markers.

| | Confusion | | Non | | |
|---|---|---|---|---|---|
| Method | Matrix | Rejection | Rejection | Surveillance | Total |
| 1 | Rejection | 38 | 0 | — | 100% |
| | Non Rejection | 3 | 59 | — | 95% |
| 2 | Rejection | 26.7 | 0 | 0 | 100% |
| | Non Rejection | 0 | 46.7 | 0 | 100% |
| | Surveillance | 0 | 0 | 26.7 | 100% |

Method 1 samples where clustered in 2 different groups (Rejection and Non Renection), Method 2, samples were clustered in 3 different categories, rejection, non rejection and surveillance. The column labelled as total represents percentage of the number of algorithm matches when a patient is a rejecter or non-rejecter for selected markers.

TABLE 8
Results after the ROC analysis when comparing the minimal combination
of key markers to achieve area under the curve (AUC) values
above 0.7. AUC represents the area under the ROC curve. AUCs
value above 0.7 indicates reliabilities above 70%.

| Combination Main Biomarkers | AUC |
|---|---|
| Angiogenin-FIBRONECT-mitDNA-MMP8 | 0.928 |
| Angiogenin-FIBRONECT-mitDNA | 0.923 |
| FIBRONECT-MIP1B-mitDNA-MMP8 | 0.895 |
| Angiogenin-MIP1B-mitDNA | 0.866 |
| Angiogenin-FIBRONECT-MMP8 | 0.847 |
| FIBRONECT | 0.847 |
| FIBRONECT-MIP1B-MMP8 | 0.842 |
| Angiogenin-FIBRONECT | 0.818 |
| Angiogenin-mitDNA | 0.813 |
| HLADR_FCM | 0.813 |
| Angiogenin-HYALURON-mitDNA | 0.809 |
| Angiogenin-MIP1B | 0.794 |
| Angiogenin-MIP1B-MMP7 | 0.78 |
| Angiogenin-HYALURON-mitDNA-MMP8 | 0.775 |
| Angiogenin-MIP1B-mitDNA-MMP8 | 0.766 |
| Angiogenin-mitDNA-MMP8 | 0.742 |
| Angiogenin-MIP1B-MMP8 | 0.732 |
| HYALURON-MIP1B | 0.713 |
| Angiogenin-MMP7 | 0.708 |

AUC represents the area under the ROC curve. AUCs value above 0.7 indicates reliabilities above 70%.

TABLE 9
Results after the ROC analysis when comparing the minimal combination
of all markers to achieve area under the curve (AUC) values
above 0.7. AUC represents the area under the ROC curve. AUC
values above 0.7 indicates reliabilities above 70%.

| Combination Biomarkers | AUC |
|---|---|
| BILE ACID-HSP70-Hyaluronan | 0.923 |
| BILE ACID-HSP70-IL6 | 0.88 |
| BILE ACID-HSP70-Nucleosomes | 0.871 |
| BILE ACID-CXCL16-HSP70 | 0.866 |
| BILE ACID-Fibronectin-Hyaluronan | 0.866 |
| Casp_3-Hyaluronan-IL18 | 0.861 |
| Cathepsin L-HSP70-IL18 | 0.852 |
| BILE ACID-Fibronectin-HSP70 | 0.847 |
| BILE ACID-HSP70-MMP2 | 0.847 |
| BILE ACID-Endostatin-Hyaluronan | 0.847 |
| Casp_8-HSP70-Hyaluronan | 0.847 |
| Casp_3-HSP70-Hyaluronan | 0.842 |
| BILE ACID-HSP70-LiveMitoc | 0.842 |
| BILE ACID-Endostatin-HSP70 | 0.837 |
| BILE ACID-DPPIV_CD26-Fibronectin | 0.837 |
| BILE ACID-HSP70-THP1_IL1 | 0.837 |
| Cathepsin L-Fibronectin-Hyaluronan | 0.837 |
| BILE ACID-DPPIV_CD26-HSP70 | 0.833 |
| Cathepsin L-Fibronectin-HSP70 | 0.833 |
| HSP70-Hyaluronan-LDH | 0.828 |
| Casp_3-DPPIV_CD26-Hyaluronan | 0.828 |
| BILE ACID-HSP70 | 0.828 |
| Casp_3-Fibronectin-Hyaluronan | 0.828 |
| Angiopoietin-2-Casp_3-Hyaluronan | 0.828 |
| BILE ACID-HSP70-IGFBP-3 | 0.828 |
| HSP70-Hyaluronan-MMP3 | 0.823 |
| BILE ACID-DPPIV_CD26-Hyaluronan | 0.818 |
| Casp_3-Endostatin-Hyaluronan | 0.818 |
| Casp_3-HSP70-Nucleosomes | 0.818 |
| Cathepsin L-Hyaluronan-IL18 | 0.818 |
| BILE ACID-Hyaluronan-MMP2 | 0.813 |
| BILE ACID-Hyaluronan-IL6 | 0.813 |
| Casp_3-Hyaluronan-LiveMitoc | 0.809 |
| BILE ACID-Hyaluronan | 0.809 |
| Angiopoietin-2-HSP70-Hyaluronan | 0.809 |
| BILE ACID-CXCL16-Hyaluronan | 0.809 |
| HSP70-Hyaluronan-URIC ACID | 0.809 |
| HSP70-LDH-VCAM-1 | 0.809 |
| DPPIV_CD26-Hyaluronan-LDH | 0.804 |
| HSP70-Hyaluronan-IL18 | 0.804 |
| BILE ACID-Hyaluronan-IGFBP-3 | 0.804 |
| Cathepsin L-Endostatin-HSP70 | 0.804 |
| DPPIV_CD26-HSP70-Hyaluronan | 0.799 |
| Fibronectin-Hyaluronan-LDH | 0.799 |
| BILE ACID-Hyaluronan-LiveMitoc | 0.799 |
| BILE ACID-Hyaluronan-Nucleosomes | 0.799 |
| BILE ACID-Hyaluronan-THP1_IL1 | 0.799 |
| Casp_3-Hyaluronan-IGFBP-3 | 0.799 |
| CXCL16-HSP70-LiveMitoc | 0.794 |
| Cathepsin L-HSP70-Hyaluronan | 0.794 |
| Hyaluronan-LDH-Nucleosomes | 0.794 |
| Hyaluronan-LDH-VCAM-1 | 0.794 |
| Casp_3-Hyaluronan | 0.794 |
| Casp_3-CXCL16-HSP70 | 0.794 |
| CCL5-HSP70-Hyaluronan | 0.794 |
| HSP70-Hyaluronan-VCAM-1 | 0.794 |
| Casp_3-CCL5-HSP70 | 0.794 |
| HSP70-LiveMitoc-MMP3 | 0.794 |
| HSP70-Hyaluronan-LiveMitoc | 0.789 |
| Hyaluronan-LDH-LiveMitoc | 0.789 |
| Endostatin-HSP70-Hyaluronan | 0.789 |
| Casp_3-Hyaluronan-THP1_IL1 | 0.789 |
| HSP70-Hyaluronan-SPECKS | 0.789 |
| CCL5-HSP70-MMP3 | 0.789 |
| HSP70-Hyaluronan-IL17A | 0.789 |
| HSP70-Hyaluronan | 0.785 |
| HSP70-Hyaluronan-MMP7 | 0.785 |
| CCL20-HSP70-Hyaluronan | 0.785 |
| Casp_3-Hyaluronan-Nucleosomes | 0.785 |
| CXCL16-HSP70-MMP3 | 0.785 |
| HSP70-Hyaluronan-IGFBP-3 | 0.785 |
| Casp_3-DPPIV_CD26-Nucleosomes | 0.785 |
| HSP70-Hyaluronan-TIMP1 | 0.785 |
| HSP70-HSP90-Hyaluronan | 0.78 |
| HSP70-Hyaluronan-THP1_IL1 | 0.78 |
| HSP70-LDH-LiveMitoc | 0.78 |
| Hyaluronan-IL18-LDH | 0.78 |
| Angiopoietin-2-HSP70-LiveMitoc | 0.78 |
| Casp_3-CCL5-Hyaluronan | 0.78 |
| CCL20-Fibronectin-HSP70 | 0.78 |
| HSP70-IL18-LiveMitoc | 0.78 |
| CXCL16-HSP70-LDH | 0.775 |
| Endostatin-Hyaluronan-LDH | 0.775 |
| BILE ACID-DPPIV_CD26-Endostatin | 0.775 |
| HSP70-IL18-LDH | 0.775 |
| Hyaluronan-LDH-THP1_IL1 | 0.775 |
| Hyaluronan-LDH-URIC ACID | 0.775 |
| Casp_3-CXCL16-Hyaluronan | 0.775 |
| HSP70-Hyaluronan-IL6 | 0.775 |
| Angiopoietin-2-Casp_3-HSP70 | 0.775 |
| Cathepsin L-CXCL16-HSP70 | 0.775 |
| Cathepsin L-Hyaluronan-Nucleosomes | 0.775 |
| HSP70-IL6-MMP2 | 0.775 |
| HSP70-LDH-Nucleosomes | 0.775 |
| Hyaluronan-LDH-MMP7 | 0.77 |
| CXCL16-HSP70-Hyaluronan | 0.77 |
| Fibronectin-HSP70-Hyaluronan | 0.77 |
| Hyaluronan-LDH | 0.77 |
| CXCL 16-Hyaluronan-LDH | 0.77 |
| HSP70-Hyaluronan-Nucleosomes | 0.77 |
| Hyaluronan-LDH-SPECKS | 0.77 |
| HSP70-LiveMitoc-MMP2 | 0.766 |
| Cathepsin L-Endostatin-Hyaluronan | 0.766 |
| HSP70-Hyaluronan-MMP2 | 0.761 |
| CXCL16-HSP70-THP1_IL1 | 0.761 |
| CCL20-HSP70 | 0.761 |
| CCL20-CXCL16-HSP70 | 0.761 |
| CCL20-Endostatin-HSP70 | 0.761 |
| DPPIV_CD26-Fibronectin-LDH | 0.761 |
| DPPIV_CD26-Hyaluronan-URIC ACID | 0.761 |
| HSP70-LDH-MMP7 | 0.756 |
| HSP70-LDH-THP1_IL1 | 0.756 |
| DPPIV_CD26-Hyaluronan-LiveMitoc | 0.756 |
| Endostatin-HSP70-LDH | 0.756 |
| Fibronectin-HSP70-LDH | 0.756 |
| Casp_3-CXCL16-DPPIV_CD26 | 0.756 |
| Casp_3-HSP70-IL18 | 0.756 |
| DPPIV_CD26-HSP70-LDH | 0.756 |
| BILE ACID-CXCL16-DPPIV_CD26 | 0.751 |
| Cathepsin L-HSP70-THP1_IL1 | 0.751 |
| HSP70-LDH | 0.751 |
| Angiopoietin-2-HSP70-THP1_IL1 | 0.751 |
| Cathepsin L-Hyaluronan | 0.751 |
| Angiopoietin-2-Hyaluronan-LiveMitoc | 0.751 |
| CCL5-HSP70-LiveMitoc | 0.751 |
| CXCL16-DPPIV_CD26-LDH | 0.751 |
| HSP70-LDH-SPECKS | 0.751 |
| Hyaluronan-LiveMitoc-URIC ACID | 0.751 |
| Casp_3-HSP70-LiveMitoc | 0.746 |
| Cathepsin L-Hyaluronan-IGFBP-3 | 0.746 |
| HSP70-LDH-URIC ACID | 0.746 |
| Fibronectin-Hyaluronan-MMP2 | 0.742 |
| Casp_3-DPPIV_CD26-LiveMitoc | 0.742 |
| CCL5-HSP70-THP1_IL1 | 0.742 |
| CXCL16-HSP70-MMP7 | 0.742 |
| DPPIV_CD26-HSP70-LiveMitoc | 0.742 |
| CCL5-DPPIV_CD26-HSP70 | 0.742 |
| DPPIV_CD26-Endostatin-LDH | 0.742 |
| Hyaluronan-IL18-MMP7 | 0.742 |
| Hyaluronan-MMP7-URIC ACID | 0.742 |
| Casp_3-HSP70-THP1_IL1 | 0.737 |
| CXCL16-HSP70-MMP2 | 0.737 |
| CXCL16-DPPIV_CD26-HSP70 | 0.737 |
| Fibronectin-HSP70-LiveMitoc | 0.737 |
| Casp_3-DPPIV_CD26-HSP70 | 0.737 |
| Cathepsin L-CXCL16-Hyaluronan | 0.737 |
| DPPIV_CD26-Hyaluronan-MMP3 | 0.737 |
| HSP70-THP1_IL1-URIC ACID | 0.737 |
| Casp_3-DPPIV_CD26-THP1_IL1 | 0.732 |
| HSP70-LiveMitoc-MMP7 | 0.732 |
| Casp_3-Fibronectin-HSP70 | 0.732 |
| HSP70-IL18-THP1_IL1 | 0.732 |
| BILE ACID-DPPIV_CD26 | 0.732 |
| HSP70-IL6-THP1_IL1 | 0.732 |
| HSP70-MMP2-Nucleosomes | 0.732 |
| HSP70-MMP7-Nucleosomes | 0.732 |
| HSP70-MMP7-URIC ACID | 0.732 |
| HSP70-LiveMitoc-THP1_IL1 | 0.727 |
| Endostatin-HSP70-LiveMitoc | 0.727 |
| Angiopoietin-2-DPPIV_CD26-Hyaluronan | 0.727 |
| Casp_3-DPPIV_CD26-Fibronectin | 0.727 |
| Casp_3-HSP70-IGFBP-3 | 0.727 |
| Cathepsin L-Hyaluronan-THP1_IL1 | 0.727 |
| CCL20-Fibronectin-Hyaluronan | 0.727 |
| CXCL16-LiveMitoc-MMP3 | 0.727 |
| DPPIV_CD26-HSP70-IL18 | 0.727 |
| HSP70-HSP90-Nucleosomes | 0.727 |
| HSP70-MMP3-THP1_IL1 | 0.727 |
| Hyaluronan-LiveMitoc-MMP2 | 0.722 |
| DPPIV_CD26-Fibronectin-Hyaluronan | 0.722 |
| HSP70-Nucleosomes-THP1_IL1 | 0.722 |
| CXCL16-HSP70-HSP90 | 0.722 |
| DPPIV_CD26-Hyaluronan-MMP7 | 0.722 |
| Casp_3-Endostatin-HSP70 | 0.722 |
| CXCL16-DPPIV_CD26-LiveMitoc | 0.722 |
| Hyaluronan-IL18-LiveMitoc | 0.722 |
| Casp_3-HSP70 | 0.718 |
| DPPIV_CD26-Hyaluronan-IL18 | 0.718 |
| HSP70-IL18-MMP7 | 0.718 |
| HSP70-THP1_IL1-VCAM-1 | 0.713 |
| Endostatin-HSP90-Hyaluronan | 0.708 |
| DPPIV_CD26-Hyaluronan-Nucleosomes | 0.708 |
| Endostatin-HSP70-MMP3 | 0.708 |
| Fibronectin-HSP70-MMP3 | 0.708 |
| Fibronectin-Hyaluronan-MMP7 | 0.708 |
| Hyaluronan-LiveMitoc-Nucleosomes | 0.708 |
| CCL20-CXCL16-Hyaluronan | 0.708 |
| CCL20-Endostatin-Hyaluronan | 0.708 |
| DPPIV_CD26-Hyaluronan-IGFBP-3 | 0.708 |
| HSP70-IGFBP-3-MMP2 | 0.708 |
| Hyaluronan-LiveMitoc-MMP3 | 0.708 |
| Fibronectin-HSP70-MMP2 | 0.703 |
| Fibronectin-Hyaluronan-LiveMitoc | 0.703 |
| HSP70-MMP2 | 0.703 |
| Casp_3-DPPIV_CD26-Endostatin | 0.703 |
| DPPIV_CD26-HSP70-MMP3 | 0.703 |
| HSP70-MMP2-SPECKS | 0.703 |

AUC represents the area under the ROC curve. AUC values above 0.7 indicates reliabilities above 70%.

1. In vitro method for predicting organ transplant rejection in a subject in need thereof which comprises: a) Assessing the concentration or expression level of the combination of biomarkers: Angiogenin and mitDNA in the organ preservation solution or in a blood sample obtained from the patient, and b) wherein the identification of a deviation or variation in the concentration or expression level with respect to a pre-established threshold level determined in control subjects, is an indication that the subject may suffer organ transplant rejection.

2. In vitro method, according to claim 1, for predicting organ transplant rejection in a subject in need thereof which comprises: a) Assessing the concentration or expression level of the following combination of biomarkers: Fibronectin, Hyaluronic acid, MIP1B, mitDNA, MMP7, MMP8, Angiogenin and ANGPT2, in the organ preservation solution or in a blood sample obtained from the patient, and b) wherein the identification of a deviation or variation in the concentration or expression level with respect to a pre-established threshold level determined in control subjects, is an indication that the subject may suffer organ transplant rejection.

3. In vitro method, according to claims 1 or 2, for predicting acute organ transplant rejection, most preferably acute liver transplant rejection, in a subject in need thereof.

4. In vitro use of the combination of biomarkers: Angiogenin and mitDNA for predicting organ transplant rejection in a subject in need thereof.

5. In vitro use, according to claim 4, of the following combination of biomarkers:
Fibronectin, Hyaluronic acid, MIP1B, mitDNA, MMP7, MMP8, Angiogenin and ANGPT2, for predicting organ transplant rejection in a subject in need thereof.

6. In vitro use, according to claim 4 or 5, for predicting acute organ transplant rejection, most preferably acute liver transplant rejection, in a subject in need thereof.

7. Kit, suitable for performing the method of any of the claims 1 to 3 which comprises reagents or tools for assessing the concentration or expression level of the combination of biomarkers: Angiogenin and mitDNA.

8. Kit, according to claim 7, suitable for performing the method of any of the claims 1 to 3 which comprises reagents or tools for assessing the concentration or expression level of the following combination of biomarkers: Fibronectin, Hyaluronic acid, MIP1B, mitDNA, MMP7, MMP8, Angiogenin and ANGPT2.

9. Use of the kit of claim 7 or 8 for predicting organ transplant rejection.

10. Use of the kit of claim 7 or 8, according to claim 9, for predicting liver transplant rejection.


SEQUENCE LISTING (2 sequences)

SEQ ID NO: 1 - DNA, 26 bp, synthetic construct
  source 1..26: mol_type = other DNA
ccccacaaaccccattactaaaccca

SEQ ID NO: 2 - DNA, 25 bp, synthetic construct
  source 1..25: mol_type = other DNA
ttcatcatgcggagatgttggatgg